(19) United States
(12) Patent Application Publication
(10) Pub. No.: US 2007/0212771 A1
(43) Pub. Date: Sep. 13, 2007

(54) Method for Preparation of a Suspension of Microorganisms

(76) Inventors: Denis Guillaud, Paladru (FR); Eloi Fontaine, Tours (FR)

Correspondence Address: FROST BROWN TODD, LLC, 2200 PNC CENTER, 201 E. FIFTH STREET, CINCINNATI, OH, 45202, US

(21) Appl. No.: 10/556,370

(22) PCT Filed: May 10, 2004
(86) PCT No.: PCT/FR04/01135

§ 371 Date: Feb. 2, 2007

(30) Foreign Application Priority Data
May 13, 2003 (FR) 03/05732

(51) Int. Cl. C12N 1/20; C12N 1/18

(52) U.S. Cl. 435/252.1; 435/254.1

(57) ABSTRACT

The invention relates to a method for preparation of a suspension of micro-organisms method for preparation of a suspension of micro-organisms in a liquid, characterised in using a sealed reservoir (1) comprising:—2 chambers communicating by means of the lower section of the reservoir, at least one inlet opening (2), for a liquid into a chamber, each chamber having at least one inlet opening (3) and one outlet opening (4), for evacuation of a gas and the reservoir further contains the microorganism in a solid form (8). The method is characterised in that the liquid is introduced by means of the inlet opening into at least one of the chambers, then a gas is alternately introduced by said inlet openings into one chamber and then the other chamber such as to generate an oscillating flow of said liquid in each chamber.

The present invention relates to a method for preparing a suspension of microorganisms in a liquid, using a sealed reservoir comprising 2 chambers into which a gas is alternately introduced so as to generate a flow of said liquid. The invention also relates to a method for seeding said microorganism.

Microorganisms are used in many industries, in particular in the agrofoods industry. They are used, inter alia, for fermenting, flavoring, refining or texturing foods, in particular dairy products or pork meat products. They are also used to protect the environment into which they are incorporated against contamination with other microorganisms, and are also used for their probiotic effects.

Depending on the applications, microorganisms are sold in dry, lyophilized, frozen form or in the form of a suspension, and are most commonly used in the form of a suspension. In the case of microorganisms in dry, lyophilized or frozen form, their use requires prior suspension. This suspension is generally carried out in tanks, mixers or reactors, the volumes of which range from 1 to 5000 liters.

The tanks or mixers currently available generally comprise a glass or stainless steel tank and an agitation system in the form of a propeller mounted at the bottom of the tank in order to stir the liquid medium.

After each mixing operation, these tanks or mixers must be washed, decontaminated and rinsed. They are optionally sterilized before each new mixing.

The operations to wash, maintain and sterilize these reactors are lengthy steps, which involve many manipulations and additional costs. In terms of cost, of time and of human means, they can represent up to 30% of the use of the reactor, which is very high.

In addition, the washing of these reactors can lead to problems of contamination if this washing is not sufficiently effective.

Furthermore, in the specific case of the suspension of microorganisms, the microorganisms must not adhere to the walls of the tank or agglomerate at the bottom of the tank due to settling out. In addition, it is essential to prevent any contamination of the suspension by the surrounding environment.

In order to satisfy the requirements of industrial companies, it has become necessary to find a method for preparing a suspension of microorganisms that makes it possible to prevent bacterial contaminations, while providing homogeneous mixing, on an industrial scale. The term “industrial scale” is intended to mean a method that makes it possible to mix large volumes and that can be used on a production line.

Thus, the problem that the present invention proposes to solve is that of providing a method for preparing a homogeneous suspension of microorganisms that prevents contaminations and is on an industrial scale.

With this aim, the invention proposes a method for preparing a suspension of microorganisms in a liquid, characterized in that a sealed reservoir (1) is used, comprising:
2 chambers that communicate by means of the lower section of the reservoir,
at least one inlet opening (2) for introducing a liquid into a chamber,
each chamber comprising at least one gas inlet opening (3) and at least one outlet opening (4) for evacuating a gas,
the reservoir also containing the microorganisms in a solid form (8),
   and characterized in that the liquid is introduced by means of the inlet opening into at least one of the chambers, then a gas is alternately introduced by means of said gas inlet openings into one chamber and then into the other chamber so as to generate an oscillating flow of said liquid in each chamber.

The invention also proposes a method of seeding, characterized in that a suspension prepared by the method mentioned above is used.

The method according to the invention has the advantage of being able to be implemented in any industries that use microorganisms, in particular the agrofoods, pharmaceutical, cosmetics or food industry, or agriculture, and also in the fields of animal nutrition, animal feedstuffs and hygiene in the broad sense, in particular body hygiene (for example, toothpastes) or industrial hygiene.

The use of the reservoir according to the invention makes it possible to provide a suspension of microorganisms which is homogeneous, and under conditions which prevent any contaminations by the outside environment.

The method according to the invention also has the advantage of reducing the phenomena of settling out and of preventing the microorganisms from concentrating at the bottom of the reservoir.

The invention will be more thoroughly understood, and other characteristics, details and advantages thereof will emerge more clearly, in the course of the explanatory description that follows, provided with reference to the attached diagrammatic drawings given only by way of examples, and in which:

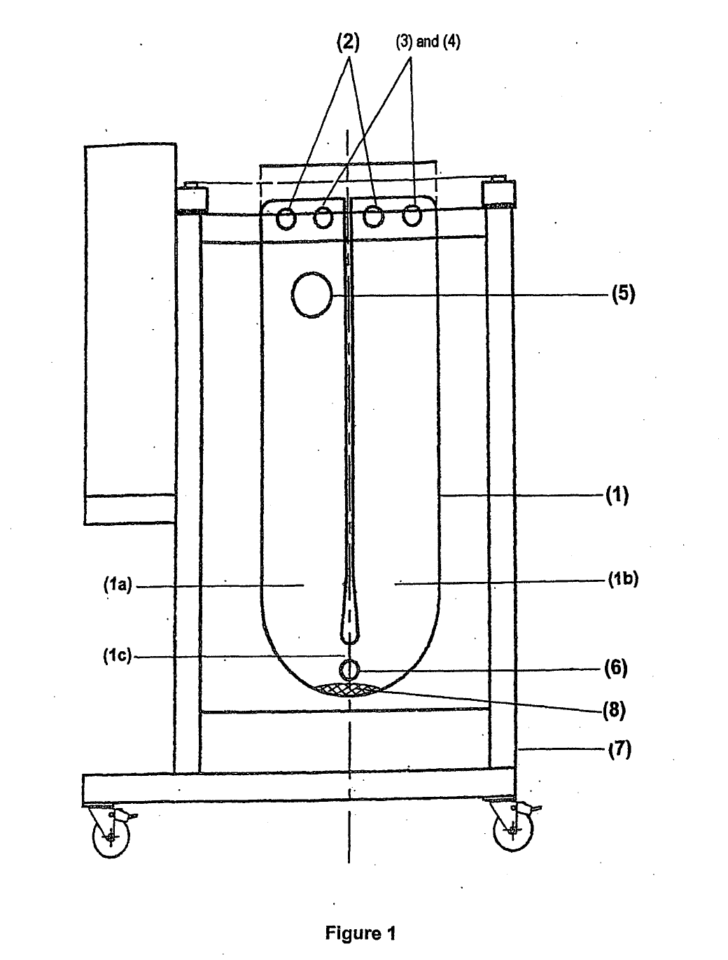

FIG. 1 is a front view of the reservoir used according to the invention;

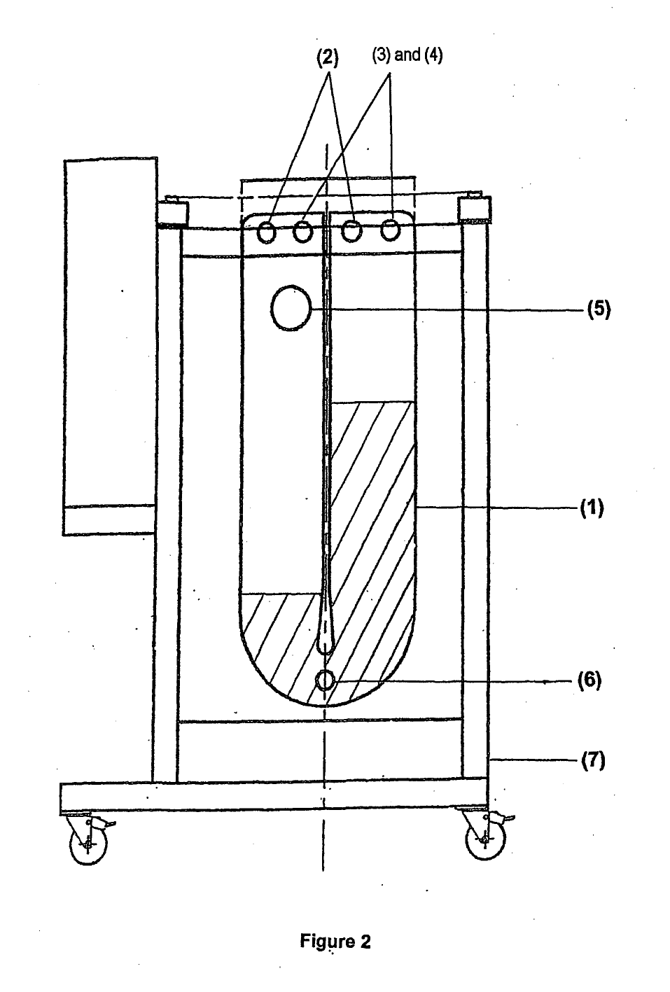

FIG. 2 is a front view of the same reservoir in the process of functioning and containing the suspension;

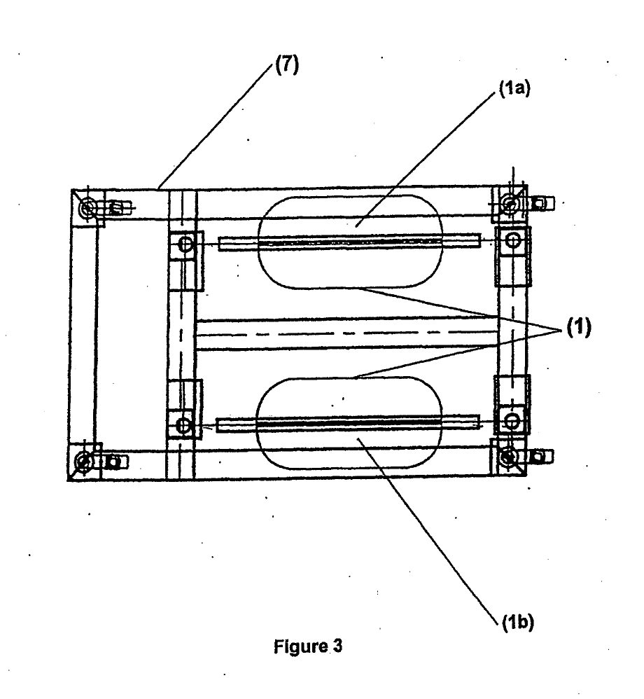

FIG. 3 is a view of this same reservoir, from above.

A subject of the present invention is first of all a method for preparing a suspension of microorganisms in a liquid, characterized in that a sealed reservoir (1), which will now be described in greater detail, is used.

This reservoir (1) comprises 2 chambers (1a) and (1b) that communicate by means of the lower section of the reservoir (1c).

At least one of the chambers also comprises at least one, preferably two, inlet opening(s) (2) for introducing a liquid into a chamber.

Each chamber comprises at least one gas inlet opening (3) and at least one outlet opening (4) for evacuating a gas.

These openings are preferably located in the upper section of the reservoir, as is seen in FIGS. 1 and 2.

These openings may be equipped with a flap, a disk, a tap, a valve or a gate, or any means intended to regulate the movement of a fluid through these openings.

Preferably, the reservoir according to the invention comprises openings (3) and (4) which allow both the inlet and the evacuation of gas alternately, as shown in FIG. 1.

The reservoir also contains the microorganisms in a solid form (8).

The chambers (1a) and (1b) of the reservoir are similar or identical. They have the same dimensions and the same capacity in terms of volume.

These chambers (1a) and (1b) may be any shape, and may have a cylindrical, circular or rectangular cross section with rounded ends as shown in FIG. 3.

Preferably, the reservoir according to the invention comprises two chambers in a substantially U-shaped arrangement.

In this particular case, each chamber (1a) and (1b) constitutes one arm of the U and the lower section (1c) of the reservoir constitutes the rounded section of the U. The expression “in a substantially U-shaped arrangement” is intended to mean an arrangement similar to a U-shape, and in which the axes of the two chambers are parallel or virtually parallel. The axes of the two chambers can form a greater angle and, in this case, the reservoir thus has a shape similar to a V-shape.

The reservoir is a sealed pocket, advantageously sealed in the upper section of the reservoir, preferably in the top section of the openings.

The reservoir according to the invention may advantageously be a single pocket folded back on itself so as to form 2 distinct chambers, (1a) and (1b), as shown in FIGS. 1 and 2. In this case, the sealed reservoir will comprise 2 chambers in a substantially U-shaped arrangement, and in which the arms of the U are very close, or even in contact, as can be seen in FIG. 1.

The preferred reservoir, for carrying out the method according to the invention, may be a flexible or supple sealed reservoir, in particular made of plastic. It is also possible to envision a rigid sealed reservoir, in particular made of stainless steel or rigid plastic.

This reservoir preferably consists of a flexible or supple material such as, for example, polypropylene, polyester, polyamide or cellulose, or any other flexible material compatible with food products. The reservoir is preferably made of polyethylene.

A flexible material will be used in the case of a reservoir that is folded up on itself.

The reservoir (1) according to the invention may comprise more particularly at least one filling opening (5) for introducing the microorganisms in the solid phase. In particular, it makes it possible to introduce a powder into the reservoir. The powder introduced is preferably a powder containing microorganisms or a powder of microorganisms to be suspended in a liquid.

It should be noted that the microorganism in the solid form (8) may also comprise polysaccharides, enzymes, minerals, proteins, sugars, nitrogenous compounds, growth factors and any compounds usually present in a mixture with microorganisms.

The reservoir (1) according to the invention may comprise at least one opening (6) for emptying of said reservoir, that makes it possible to empty the reservoir.

The opening (6) is preferably located in the lower section of the reservoir.

The openings (5) and (6) above may be equipped with a flap, a disk, a tap, a valve or a gate or any means intended to regulate the movement of a solid or of a fluid through these openings.

The capacity of the reservoir according to the invention is variable, and in particular it can be between 5 liters and 100 liters, preferably between 10 liters and 50 liters. In addition, for correct functioning of the reservoir, the section (1c) should have a vertical cross section of at least 15 cm.

The term “microorganisms” is intended to mean, for example, yeast, bacteria, in particular lactic acid bacteria, and more generally all the microorganisms used in the agrofoods, pharmaceutical, cosmetics or food industry, or agriculture, and in the fields of animal nutrition, animal feedstuffs and hygiene in a broad sense, in particular body hygiene (for example, toothpaste) or industrial hygiene. These microorganisms are used alone or as mixtures.

Among the bacteria most commonly used and which are present in ferments, mention may be made of those belonging to the Lactococcus, Streptococcus, Lactobacillus, Leuconostoc, Pediococcus, Bifidobacterium, Brevibacterium, Carnobacterium, Enterococcus, Micrococcus, Vagococcus, Staphylococcus, Bacillus, Kocuria, Arthrobacter and Corynebacterium genera.

It should be noted that the reservoir is advantageously attached, by means of its upper section, to a support bracket (7), in particular a mobile support bracket. This support bracket can be moved when it is mounted, for example, as in FIG. 1, on wheels to all the sections of the industrial system, before, after or during the industrial process.

The term “mobile support bracket” is preferably intended to mean a metal or nonmetal frame, on which the reservoir according to the invention is suspended. The reservoir is preferably suspended vertically.

When the reservoir is suspended, the microorganisms in the solid form (8) are preferably in section (1c) of the reservoir.

The reservoir for carrying out the method according to the invention may also be a disposable sealed reservoir. In particular in the case of a plastic sealed reservoir, the latter may be for single use and disposable after use.

It is also possible to envision sterilizing the sealed reservoir before the introduction of the microorganisms (8). This sterilization can be carried out, for example, by UV- or gamma-radiation.

The functioning of the reservoir will now be described.

First of all, the reservoir is filled with the microorganisms in the form of a solid (8). It should be noted, at this stage, that it is also possible to add other additional compounds, which are described above (polysaccharides, enzymes, minerals, proteins, sugars, nitrogenous compounds, growth factors or any compounds usually present in a mixture with microorganisms), in any form.

During filling, the reservoir may be in unfolded or folded form, or in the suspended position. If the reservoir was in the unfolded form for filling, it is subsequently folded on itself in a substantially U-shape and suspended from the support bracket.

The method according to the invention preferably uses a reservoir that can be used for suspending microorganisms in a liquid medium. This suspending is carried out by virtue of the agitation of said liquid in the presence of the microorganism in the solid form, in particular in the form of a powder, in the reservoir.

At least one of the inlet openings (2) allows said liquid to be introduced into the sealed reservoir.

Advantageously, the liquid introduced is an aqueous medium, and more particularly water, water containing stabilizers, a milk medium or milk-based medium, or fruit juices.

The aqueous medium is sterilized beforehand; it is preferably filtered through a membrane of at most 0.45 μm, more particularly at most 0.22 μm. It should be noted that tap water can be used.

Advantageously, the temperature of the aqueous medium as it is introduced into the sealed reservoir is between 5° C. and 50° C., preferably between 8° C. and 20° C.

The method according to the invention uses a gas which is introduced into at least one of the chambers by means of at least one of the openings (3) that allows gas to be introduced into the reservoir (1).

For the purposes of the present invention, the term “gas” also includes air.

Air or a gas that is not involved in the respiration and/oxidation of microorganisms, of ferments and of bacteria is advantageously used.

The gas injected may be a chemically and biologically inert gas; preferably, air, argon, more particularly nitrogen or carbon dioxide, or mixtures thereof, is injected.

The term “biologically inert gas” is intended to mean a gas that is not involved in the multiplication and degradation of microorganisms.

First of all, gas is introduced into a chamber, for example the chamber (1a). This introduction causes the level of the liquid in the chamber (1a) to drop. The introduction of gas into the chamber (1a) via the opening (3) is immediately followed by the evacuation, by means of the opening (4), of the gas that is above the level of said liquid present in the chamber (1b), which causes the level of the liquid in the chamber (1b) to rise. Gas is then introduced, by means of the opening (3), into the chamber (1b), which causes the liquid to be pushed vertically. This push results in the liquid being transferred from the chamber (1b) to the chamber (1a) and is immediately followed by the evacuation of the gas that is above the level of said liquid present in the chamber (1a).

The agitation is carried out by successive injection of gas into each chamber, which generates a vertical push applied to the liquid, which will enable the suspension to be transferred from one chamber of the reservoir to the other.

FIG. 2 shows a reservoir according to the invention in the process of functioning, where the liquid is transferred from the first chamber to the second chamber under the effect of the vertical push.

The introduction of gas into either one of the chambers is preferably initiated automatically by virtue of 2 optical cells located on either side of the chambers. These cells trigger the introduction of gas when the optical beam is, for example, broken.

The gas pressure in the sealed reservoir, during the agitation, is less than 5 bar, more particularly less than 1 bar.

The gas pressure is preferably determined as a function of the performance levels of the material used to manufacture the reservoir.

The gas can also be injected with regular time periods between each introduction of gas. Preferably, the time period between each introduction of gas is between 0.5 minutes and 30 minutes.

The agitation enables the microorganisms to be suspended in the liquid medium.

When the aqueous suspension of microorganisms is produced, it is possible to empty the sealed reservoir aseptically by means of the emptying opening (6).

This emptying means is advantageously placed at the bottom of the reservoir, but it can also be placed at other places on the reservoir, according to the various applications.

This emptying is carried out by injection of gas or of air into the sealed reservoir by means of at least one opening (3), or by gravity of the aqueous suspension of microorganisms.

According to an advantageous application, the method according to the invention uses said reservoir to seed a dairy medium with a suspension of microorganisms.

A subject of the invention is also a method of seeding, characterized in that a suspension of microorganisms prepared by the method described above is used.

More particularly, it is a method of seeding a dairy medium, but other media, mentioned below, can be envisaged.

For example, the suspension of microorganisms may be a ferment which can be introduced into the medium to be seeded, in particular a milk medium, so as to trigger the fermentation of said milk medium, and/or the texturing of this medium.

The seeding of a milk medium with a suspension of microorganisms is advantageously carried out at a flow rate of between 10 ml/min and 1000 ml/min, preferably between 100 ml/min and 500 ml/min. The flow rate is regulated by a pump or by the injection of gas into the reservoir.

This seeding is carried out at a temperature advantageously of between 5° C. and 50° C., preferably between 8° C. and 45° C.

The seeding of the milk medium with the suspension of microorganisms is carried out over a period that can extend up to 72 hours, more particularly over a period that can extend up to 48 hours, preferably over a period that can extend up to 24 hours.

It is possible to seed the milk medium by means of the simultaneous or staggered use of several reservoirs according to the invention, on the milk line.

A first variant of the method in terms of the seeding step consists in seeding the milk medium to be treated, with the suspension of ferments, in a single step. This is carried out by completely emptying the sealed reservoir(s) in one go. This can be batch (a single reservoir) or multibatch (several reservoirs) seeding.

A second variant of the method in terms of the seeding step consists in seeding the milk medium to be treated, with the suspension of microorganisms, continuously.

A third variant of the method in terms of the seeding step consists in seeding the milk medium to be treated, with the suspension of ferments, discontinuously.

The term “discontinuously” is intended to mean a seeding cycle carried out in the following way: the milk medium to be treated is seeded for a period of time, the seeding is then stopped, and then the seeding is resumed, this being carried out for several cycles.

In the context of this third variant, the seeding of the milk medium to be treated with the suspension of microorganisms is carried out at a flow rate of between 10 ml/min and 1000 ml/min, preferably between 100 ml/min and 500 ml/min, said seeding being performed at regular or irregular intervals of between 1 minute and 600 minutes.

The variants described above can also be implemented in the case of the seeding of a meat medium or of a salt meat product, for example a pork meat product, but also and more generally in the agrofoods, pharmaceutical, cosmetics or food industry, or agriculture, and in the fields of animal nutrition, pet foods and toothpastes or dental compositions.

The advantage of implementing the method according to the invention by means of the seeding reservoir as described above is that of performing a direct, sterile, standardized seeding at ambient temperature, that can be adjusted to each type of production and that guarantees the bacteriological quality.

Another advantage of the implementation of the method according to the invention by means of the seeding reservoir as described above is that of simplifying and making reliable the step consisting in seeding the lactic acid ferment.

Another advantage of the present invention is that of providing a disposable reservoir, which reduces the risks of contamination by other microorganisms subsequent to re-use of the reservoirs. In addition, this avoids tank maintenance, limits investment and eliminates the step consisting in preparing a leaven.

1. A method for preparing a suspension of microorganisms in a liquid, characterized in that a sealed reservoir (1) is used, comprising:
2 chambers that communicate by means of the lower section of the reservoir,
at least one inlet opening (2) for introducing a liquid into a chamber,
each chamber comprising at least one gas inlet opening (3) and at least one outlet opening (4) for evacuating a gas,
the reservoir also containing the microorganisms in a solid form (8),
and characterized in that the liquid is introduced by means of the inlet opening into at least one of the chambers, then a gas is alternately introduced by means of said gas inlet openings into one chamber and then into the other chamber so as to generate an oscillating flow of said liquid in each chamber.

2. The method as claimed in claim 1, characterized in that a sealed reservoir comprising 2 chambers in a substantially U-shaped arrangement is used.

3. The method as claimed in claim 2, characterized in that a sealed reservoir that is a single pocket folded back on itself so as to form the 2 chambers (1a) and (1b) is used.

4. The method as claimed in claim 1, characterized in that flexible or supple sealed reservoir (1) is used.

5. The method as claimed in claim 1, characterized in that a reservoir comprising at least one filling opening (5) for introducing the microorganisms in the form of a solid (8) into the reservoir is used.

6. The method as claimed in claim 1, characterized in that a reservoir comprising at least one opening (6) for emptying said reservoir is used.

7. The method as claimed in claim 1, characterized in that gases such as selected from air, argon, nitrogen, carbon dioxide, or mixtures thereof, are used.

8. The method as claimed in claim 1, characterized in that a sealed reservoir attached to a support bracket (7) is used.

9. A method of seeding, characterized in that a suspension prepared by means of the method as claimed in claim 1 is used.

10. The method as claimed in claim 4, characterized in that the flexible sealed reservoir is made from plastic.

11. The method as claimed in claim 8, characterized in that the support bracket is a mobile support bracket.
